(19) United States
(12) Patent Application Publication
(10) Pub. No.: US 2020/0046290 A1
(43) Pub. Date: Feb. 13, 2020

(54) ELECTRODE INSTRUMENT AND BIOLOGICAL INFORMATION MEASURING DEVICE

(71) Applicant: Sharp Kabushiki Kaisha, Sakai City, Osaka (JP)

(72) Inventors: Hiroshi SAKAYA, Sakai City (JP); Tetsuya HAYASHI, Sakai City (JP); Azusa NAKANO, Sakai City (JP)

(21) Appl. No.: 16/485,825

(22) PCT Filed: Feb. 20, 2018
(86) PCT No.: PCT/JP2018/006060

§ 371 Date: Aug. 14, 2019

(30) Foreign Application Priority Data
Feb. 27, 2017 (JP) 2017-035076

(51) Int. Cl. A61B 5/00

(52) U.S. Cl. CPC A61B 5/6831; A61B 2503/40; A61B 2562/046; A61B 2562/164

(57) ABSTRACT

In order to increase the chances of acquiring biological information from a haired living organism, an electrode instrument includes: a belt configured to fit the electrode instrument to a living organism; a projection projecting from the belt; and an electrically conductive cloth disposed on a surface of the projection or in a vicinity thereof to function as a part of an electrode.

### TECHNICAL FIELD

The following disclosure relates to electrode instruments and biological information measuring devices and in particular to electrode instruments and biological information measuring devices that are suited to the measurement of biological information on, for example, a haired animal.

### BACKGROUND ART

It is widely recognized that everyday health management plays an important role in the prevention and treatment of lifestyle diseases. A common technique for everyday health management is to record biological information such as electrocardiographic waveforms over an extended period of time and analyze changes in the biological information.

Patent Literature 1 discloses a wearable medical sensor system for measuring biological information (electrocardiograms), which is an example technique of acquiring biological information over an extended period of time. The wearable medical sensor system described in Patent Literature 1 includes a piece of clothing and an elastic bounding layer attached on the inner side of the piece of clothing. The elastic bounding layer has a plurality of electrodes attached thereon and has a contractility greater than the contractility of the clothing and a stretchability greater than the stretchability of the clothing. Thus, when a user wears the wearable medical sensor system, the elastic bounding layer attaches and fixes each electrode to a predetermined measuring position on the user's body.

Patent Literature 2 discloses a wearable electrode including a projection at least on a part thereof that can come into contact with a living organism. The projection is provided by a member that has a higher friction coefficient than the member that provides the electrode. The wearable electrode is hence capable of stably detecting biological signals even during vigorous physical exercise.

Awareness is now growing among animal owners that everyday health management is just as important in companion animals as in humans (owners of animals). There is an increasing need for owners to readily measure biological information on their companion animals.

If a dry electrode is used in electrocardiogram measurement on an animal (e.g., a dog), it is difficult to stably acquire signals because of the presence of fur. This problem may be addressed by Patent Literature 3 disclosing an electrocardiogram electrode, for use with animals, including many highly conductive metal needles implanted in one of the surfaces of a round metal plate like needles on a pin frog. In the electrode for use with animals disclosed in Patent Literature 3, the tips of the metal needles arranged like those on a pin frogs can reach the body surface of a long haired animal.

### CITATION LIST

### Patent Literature

Patent Literature 1: Japanese Unexamined Patent Application Publication, Tokukai, No. 2016-112384 (Publication Date: Jun. 23, 2016)

Patent Literature 2: Japanese Unexamined Patent Application Publication, Tokukai, No. 2016-36642 (Publication Date: Mar. 22, 2016)

Patent Literature 3: Japanese Unexamined Patent Application Publication, Tokukaihei, No. 5-200008 (Publication Date: Aug. 10, 1993)

### SUMMARY OF INVENTION

### Technical Problem

The electrode for use with animals disclosed in Patent Literature 3, however, is provided with metal needles. For this reason, the animal on which measurement is to be made may not like to wear the electrode, and the electrode could injure the animal's skin. The electrode is not preferable for use in acquisition of biological information.

The present invention, in an aspect thereof, has an object to provide an electrode instrument and a biological information measuring device that increase the chances of acquiring biological information from a haired living organism.

### Solution to Problem

The present invention, in an aspect thereof, is directed to an electrode instrument that acquires biological information, the instrument including: a fitting device configured to fit the electrode instrument to a living organism; a projection projecting from the fitting device; and electrically conductive fiber disposed on a surface of the projection or in a vicinity thereof to function as a part of an electrode.

### Advantageous Effects of Invention

The present invention, in an aspect thereof, can advantageously increase the chances of acquiring biological information from a haired living organism.

### BRIEF DESCRIPTION OF DRAWINGS

FIG. 1 is a block diagram of a configuration of a biological information measuring device in accordance with Embodiment 1 of the present invention.

FIG. 2 is an illustration of an appearance of a measuring unit and a signal processing unit that are included in the biological information measuring device.

FIG. 3 is an illustration of a dog wearing the measuring unit and the signal processing unit as viewed from a side of the dog.

FIG. 4 is a perspective view of a structure of an electrode instrument included in the biological information measuring device.

FIG. 5 illustrates the structure of the electrode instrument included in the biological information measuring device, (a) of FIG. 5 being a cross-sectional view of the electrode instrument taken along a direction in which a belt included in the biological information measuring device is extended and (b) of FIG. 5 being a cross-sectional view of the electrode instrument taken parallel to a plane perpendicular to the direction in which the belt is extended.

FIG. 6 is a diagram of an example hardware configuration of an information processing terminal included in the biological information measuring device.

Each portion (a) and (b) of FIG. 7 is a cross-sectional view of a structure of an electrically insulating member as a variation example of an electrically insulating member included in the electrode instrument.

FIG. 8 illustrates a structure of an electrode instrument as a variation example of the electrode instrument of Embodiment 1, (a) of FIG. 8 being a cross-sectional view of the electrode instrument taken along the direction in which the belt is extended, (b) of FIG. 8 being a cross-sectional view of the electrode instrument taken parallel to a plane perpendicular to the direction in which the belt is extended, and (c) of FIG. 8 being a cross-sectional view of a structure of a cap included in the electrode instrument.

FIG. 9 illustrates effects of the electrode instrument, (a) of FIG. 9 showing an electrocardiogram drawn based on a cardiac electrical activity signal acquired by an electrode instrument including electrically insulating members that have no projections and (b) of FIG. 9 showing an electrocardiogram drawn based on a cardiac electrical activity signal acquired by an electrode instrument in accordance with an embodiment of the present invention.

FIG. 10 is a side view of an electrode instrument in accordance with Embodiment 2 of the present invention.

FIG. 11 is a cross-sectional view of the electrode instrument taken parallel to a plane perpendicular to the direction in which the belt is extended.

FIG. 12 is a cross-sectional view of an electrode instrument in accordance with Embodiment 3 of the present invention.

FIG. 13 illustrates a structure of an electrode instrument in accordance with Embodiment 4 of the present invention, (a) of FIG. 13 being a side view of the electrode instrument and (b) of FIG. 13 being a cross-sectional view of a structure of a projection included in the electrode instrument.

### DESCRIPTION OF EMBODIMENTS

### Embodiment 1

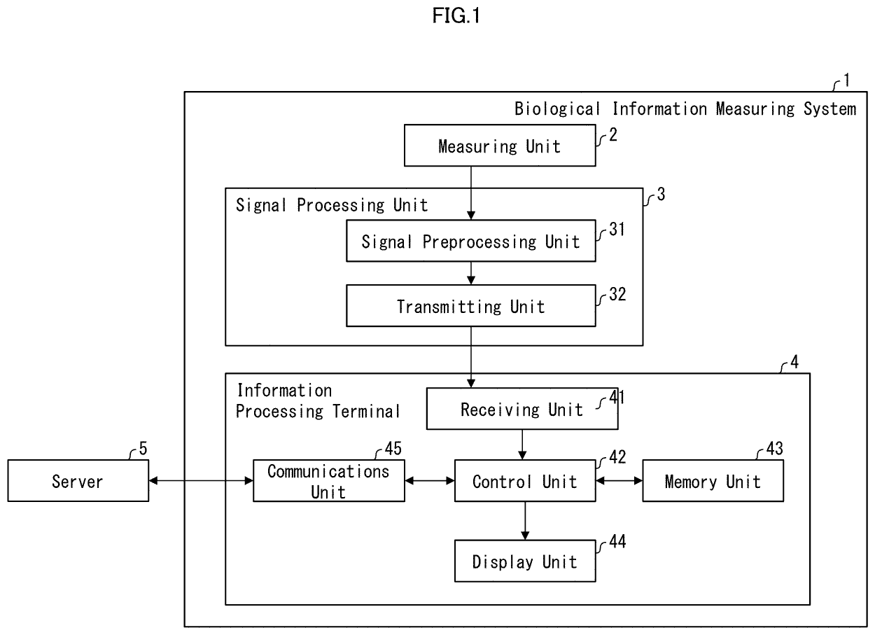

The following will describe a biological information measuring system 1 in detail in accordance with Embodiment 1 of the present invention with reference to FIGS. 1 to 6. The biological information measuring system 1 will be described in relation to measurement of biological information (electrocardiogram) on a dog. However, the animal (living organism) on which measurement is to be made for biological information thereof is not necessarily a dog and may be, for example, a human, a cat, a rabbit, a horse, a cow, or another like animal. The biological information measuring system 1 does not necessarily measure an electrocardiogram and may alternatively measure, for example, body temperature, pulse wave, perspiration rate, heart rate, body fat, or other biological information.

FIG. 1 is a block diagram of a configuration of the biological information measuring system 1. Referring to FIG. 1, the biological information measuring system 1 includes: a measuring unit 2 to be fitted to a living organism to acquire biological information from the living organism; a signal processing unit 3 for processing biological information outputted by the measuring unit 2; and an information processing terminal 4 connected to the signal processing unit 3 in a communicable manner. The signal processing unit 3 generates digital biological information (cardiac electrical activity data) from an analog signal (cardiac electrical activity signal) that represents biological information. The information processing terminal 4 processes the biological information data outputted by the signal processing unit 3 and may be, for example, a smartphone, a mobile phone, a tablet terminal, a computer (e.g., a personal computer), or a smart watch.

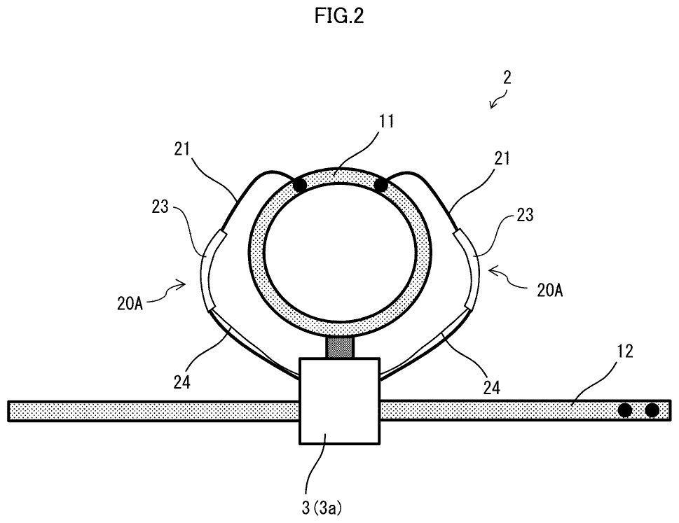

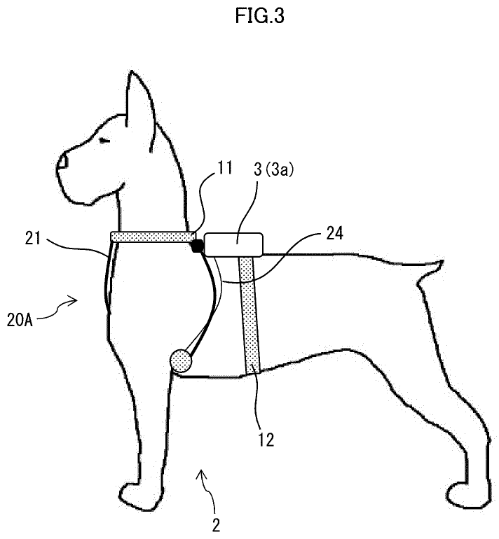

The measuring unit 2 will be now described with reference to FIGS. 2 and 3. FIG. 2 is an illustration of an appearance of the measuring unit 2 and the signal processing unit 3. FIG. 3 is an illustration of a dog wearing the measuring unit 2 and the signal processing unit 3 as viewed from a side of the dog.

Referring to FIGS. 2 and 3, the measuring unit 2 includes a neck belt 11, a trunk belt 12, and two electrode instruments 20A for acquiring the dog's biological information (cardiac electrical activity signal).

The neck belt 11 and the trunk belt 12 are placed around the dog's neck and trunk respectively to fit the measuring unit 2 and the signal processing unit 3 to the dog. The neck belt 11 and the trunk belt 12 are connected to a housing 3a of the signal processing unit 3.

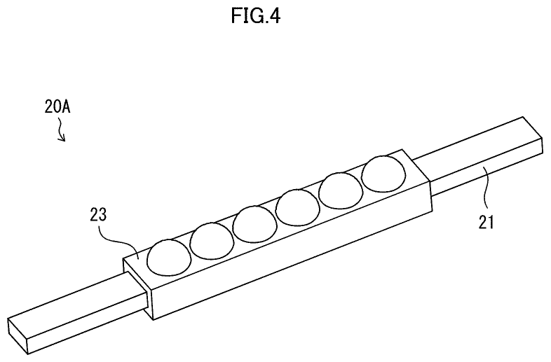

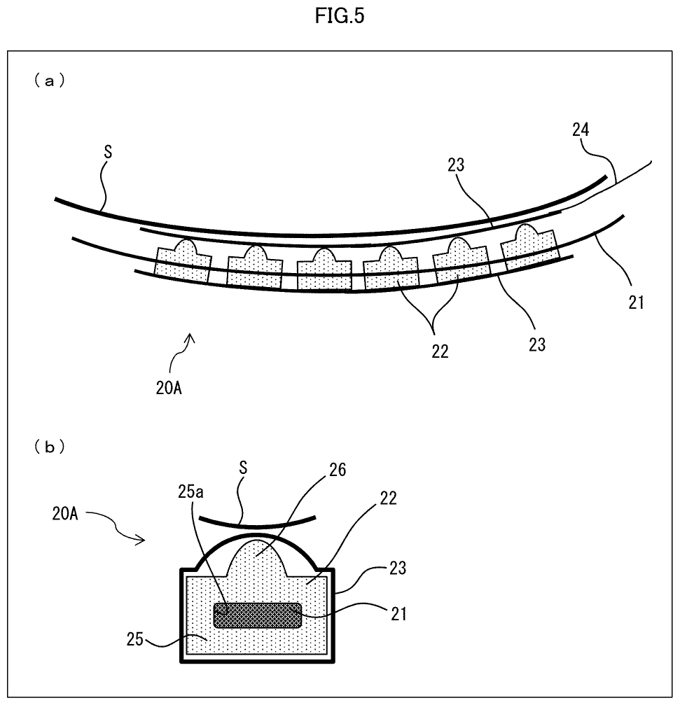

A description will be now given of the electrode instruments 20A with reference to FIGS. 2 to 5. FIG. 4 is a perspective view of a structure of the electrode instrument 20A. FIG. 5 illustrates the structure of the electrode instrument 20A, (a) of FIG. 5 being a cross-sectional view of the electrode instrument 20A taken along a direction in which a belt 21 is extended and (b) of FIG. 5 being a cross-sectional view of the electrode instrument 20A taken parallel to a plane perpendicular to the direction in which the belt 21 is extended.

As shown in FIGS. 2 to 5, the electrode instrument 20A includes the belt (fitting device) 21, electrically insulating members 22, an electrically conductive cloth (electrically conductive member) 23, and a cable 24. The combination of the electrically insulating members 22 and the electrically conductive cloth 23 can be described as providing electrodes.

The belt 21 is for fitting the electrode instrument 20A to a dog. The belt 21 is a rubber string and has an end thereof connected to the neck belt 11 and another end thereof connected to the housing 3a of the signal processing unit 3. The belt 21 is fitted to the dog so as to pass under either the left or right front armpit (over the inner side of the forelimbs) of the dog. The belt 21 needs only to be made of a material that has such stretchability that a projection 26 and the electrically conductive cloth 23 can be pressed onto the dog's skin. The belt 21 may be made of a cloth strip or other non-rubber material.

The electrically insulating members 22 provide electrode bodies and are made of an electrically insulating material (e.g., a synthetic resin). The electrically insulating members 22 are preferably made of a material that is sufficiently hard to be undeformable when the animal on which measurement is to be made changes its posture. Each electrically insulating member 22, as shown in (b) of FIG. 5, includes: a rectangular parallelepiped base 25 having formed therein a through hole 25a through which the belt 21 is passed; and the projection 26 projecting from the base 25. The projection 26 is circular in a cross-section taken perpendicular to the projection direction thereof and has a curved top (not shaped like a sharp needle tip). The base 25 of the present embodiment is rectangular in a cross-section taken perpendicular to the direction in which the belt 21 is extended. Alternatively, in another aspect of the present disclosure, the base 25 may be, for example, circular, elliptical, triangular, or otherwise polygonal or generally polygonal with curved, non-angled corners (chamfered polygonal shape). The projection 26 has a projecting height above the base 25 that may be specified in a suitable manner in view of the physique of the animal on which measurement is to be made. When measurement is to be made on a dog as in the present embodiment, the projection 26 preferably has a projecting height of 2 to 10 mm. These projecting heights allow continuous acquisition of a cardiac electrical activity signal from the dog even if the dog changes its posture. The electrically insulating member 22 may be coated with a lubricant or otherwise treated so as to have a small friction coefficient on the peripheral surface thereof that comes into contact with the electrically conductive cloth 23 (which will be described later in detail) and on the inner surface of the through hole 25a. This treatment enables the electrically insulating member 22 to easily change location relative to the belt 21 and the electrically conductive cloth 23. That can in turn position the electrically insulating member 22 in a more suitable location and reduce friction on the body surface of the animal on which measurement is to be made.

The electrode instrument 20A includes six electrically insulating members 22 in each belt 21 in such a manner that the projections 26 are lined up forming an array of them. The belt 21 does not necessarily include six electrically insulating members 22 and may include an appropriate number of electrically insulating members 22 that suits, for example, the size and physique of the animal on which measurement is to be made. The array of the six electrically insulating members 22 has a length (i.e., the distance from the electrically insulating member 22 located on one end of the array to the electrically insulating member 22 located on the other end thereof) that may be specified in a suitable manner in view of, for example, the size and physique of the animal on which measurement is to be made. For instance, this array length may be set to 4 to 7 cm for small-sized dogs, 6 to 9 cm for medium-sized dogs, and 8 to 12 cm for large-sized dogs. In addition, although FIG. 4 shows a single array of projections 26, each electrically insulating member 22 may alternatively have a plurality of projections 26 projecting in the same direction with the projections 26 being arranged in two or more arrays. Additionally, every two adjacent projections 26 are separated by an interval that may be specified in a suitable manner in view of the physique of the animal on which measurement is to be made. The intervals may be equal as shown in (a) of FIG. 5 or different from each other.

The electrically conductive cloth 23 includes electrically conductive, interwoven threads (electrically conductive fiber) which are threads plated with silver or a like high-conductivity metal. The electrically conductive cloth 23 is hence electrically conductive. The electrically conductive cloth 23 may be alternatively described as being electrically conductive fiber that is shaped as a piece of cloth. The electrically conductive cloth 23 can be electrically connected to the signal processing unit 3 via the cable 24. The electrically conductive thread may be, besides these examples: a thread of metal such as gold, silver, copper, or stainless steel; a thread of an electrically conductive polymer such as polyaniline or polyacetylene; a silver-plated nylon thread formed of a multi-filament which is a bundle of silver-plated nylon filaments; a filament thread or spun yarn (twisted yarn) of acrylic fiber, nylon fiber, or polyester fiber containing copper sulfide and nickel; or core yarn, yarn doubling, folded yarn, blended yarn, or spun yarn (twisted yarn) of an electrically conductive thread and a non-electrically conductive cotton, acrylic, nylon, or polyester thread.

The electrically conductive cloth 23 is disposed on the surface of each projection 26 and in a vicinity thereof and functions as an electrode for the electrode instrument 20A. Specifically, the electrically conductive cloth 23 covers the belt 21 and the six electrically insulating members 22. The electrically conductive cloth 23 may be shaped like a tube covering the circumference of the belt 21 and the six electrically insulating members 22. There may be further provided an adhesive member between the electrically conductive cloth 23 and the electrically insulating members 22. The electrically conductive cloth 23 preferably has a resistance of less than or equal to a few Ω/cm². These resistance values can increase sensitivity in the detection of the dog's cardiac electrical activity signals, which enables acquisition of a cardiac electrical activity signal with an amplitude that can be processed by the signal processing unit 3 (detailed later).

The electrically conductive cloth 23 is formed by interweaving only electrically conductive threads. Alternatively, the electrically conductive cloth, in an embodiment of the present invention, may be formed by interweaving electrically conductive threads and non-electrically conductive threads, for example, for adhesion of the cloth to the animal skin and to reduce damage to the animal skin. Examples of the non-electrically conductive thread include cotton, acrylic, nylon, and polyester threads.

The cable 24 is a conductive wire for transmitting the dog's cardiac electrical activity signal acquired by the electrically conductive cloth 23 to the signal processing unit 3. The cable 24 has an end thereof connected to the electrically conductive cloth 23 and the other end thereof connected to the signal processing unit 3, thereby electrically connecting the electrically conductive cloth 23 to the signal processing unit 3. The cable 24 may be either attached to the belt 21 or interwoven into the belt 21. This structure can prevent the dog from getting its forelimb caught by the cable 24 and hence breaking the cable 24. The cable 24 is preferably shielded. The shielding can reduce noise in the cardiac electrical activity signal.

In the electrode instrument 20A, the electrically insulating members 22 are positioned in the belt 21 in such a manner that when the electrode instrument 20A is fitted to the dog, the projections 26 come into contact with the front armpit of the dog with the electrically conductive cloth 23 intervening therebetween. Since the belt 21 is made of rubber, the projections 26 and the electrically conductive cloth 23 are pressed onto the dog's skin due to the stretchability of the belt 21 when the electrode instrument 20A is fitted to the dog. Therefore, the projections 26 separate the dog's hairs so that the electrically conductive cloth 23 can be placed in electrical contact with the surface of the dog's skin. To easily separate the dog's hairs, the projections 26 are preferably hard enough that the projections 26 do not deform when pressed onto the animal. Specifically, the projections 26 preferably have a Shore hardness of at least 40.

The disposition of the electrically conductive cloth 23 on the surfaces of the projections 26 inhibits the projections 26 from coming into direct contact with the surface of the dog's skin. Hence, measuring cardiac electrical activity over an extended period or with the dog being allowed to move will unlikely injure the dog's skin. Additionally, the electrically conductive cloth 23, which is soft, gives less damage to the dog's skin when the electrically conductive cloth 23 comes into contact with the surface of the dog's skin. The electrically conductive cloth 23 also includes electrically conductive, interwoven threads (electrically conductive fiber) and for this reason has small voids on the surface thereof. The dog's hairs go into these small voids when the electrically conductive cloth 23 is pressed onto the dog's body surface, which facilitates the contact of the electrically conductive cloth 23 to the dog's skin. The electrode instrument 20A is therefore more dog-friendly and more likely to successfully acquire information on the dog's cardiac electrical activity than electrodes formed of metal with a smooth surface.

Since the electrode instrument 20A includes the six projections 26 arranged in an array in the belt 21, the six projections 26 are positioned on the skin surface of the dog and come into contact with the dog's body surface when the electrode instrument 20A is fitted to the dog. This structure enhances adhesion of the electrically conductive cloth 23 to the skin surface of the dog and also increases contact area between the electrically conductive cloth 23 and the dog's body surface.

Next, the signal processing unit 3 will be described with reference to FIGS. 1 to 3. The signal processing unit 3, as shown in FIGS. 2 and 3, includes the housing 3a containing circuitry (not shown) functioning as a signal preprocessing unit 31 and a transmitting unit 32 (see FIG. 1) of the signal processing unit 3. The signal preprocessing unit 31 includes a plurality of filters (not shown) and amplifiers (not shown) to generate cardiac electrical activity data from cardiac electrical activity signals acquired from the dog by the electrically conductive cloth 23. The transmitting unit 32 outputs the cardiac electrical activity data generated by the signal preprocessing unit 31 to the information processing terminal 4 over a wireless link.

Now, the information processing terminal 4 will be described with reference to FIG. 1. Referring to FIG. 1, the information processing terminal 4 includes a receiving unit 41, a control unit 42, a memory unit 43, a display unit 44, and a communications unit 45.

The receiving unit 41 receives the cardiac electrical activity data outputted by the signal processing unit 3 and outputs the received cardiac electrical activity data to the control unit 42.

The communications unit 45 communicates with external devices (e.g., a server) over a communication link such as the Internet. The communications unit 45 acquires, for example, an electrocardiogram stored in a server 5.

The control unit 42 analyzes the cardiac electrical activity data outputted by the receiving unit 41 and draws an electrocardiogram (electrocardiographic waveform). The control unit 42 stores the drawn electrocardiogram in the memory unit 43. The control unit 42 may control the display unit 44 to display the drawn electrocardiogram. The control unit 42 may transmit the drawn electrocardiogram to the external server 5 via the communications unit 45. The control unit 42 may statistically process a plurality of electrocardiograms stored in the memory unit 43 or the server 5 and control the display unit 44 to display results of the processing.

The control blocks of the information processing terminal 4 (particularly, the control unit 42) may be implemented by logic circuits (hardware) fabricated, for example, in the form of an integrated circuit (IC chip) and may be implemented by software executed by a CPU (central processing unit).

In the latter form of implementation, the information processing terminal 4 includes, among others: a CPU that executes instructions from programs or software by which various functions are implemented; a ROM (read-only memory) or like storage device (referred to as a “storage medium”) containing the programs and various data in a computer-readable (or CPU-readable) format; and a RAM (random access memory) into which the programs are loaded. The computer (or CPU) then retrieves and executes the programs contained in the storage medium, thereby achieving the object of the present invention. The storage medium may be a “non-transient, tangible medium” such as a tape, a disc, a card, a semiconductor memory, or programmable logic circuitry. The programs may be supplied to the computer via any transmission medium (e.g., over a communications network or by broadcasting waves) that can transmit the programs. The present invention encompasses data signals on a carrier wave that are generated during electronic transmission of the programs.

The provision of the communications unit 45 (wired or wireless communications circuit) in the information processing terminal 4 enables the transmission of results of measurement (calculation) to another communication device (external communication device, server 5). When this is the case, the results of measurement, once received, may be stored, compared with other measured values, or notified to a user such as the owner. The other communication device may be, for example, a smartphone, a mobile phone, a tablet terminal, a computer (e.g., a personal computer), or a smart watch.

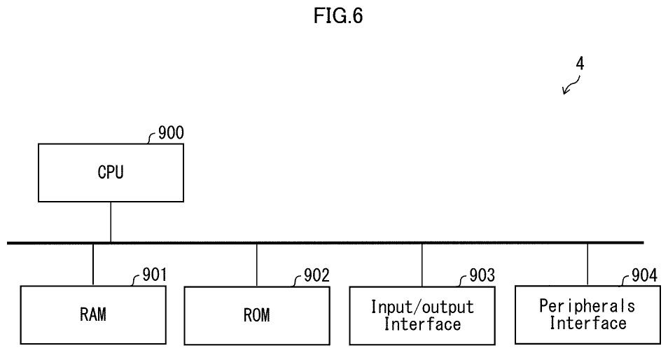

A description will be now given of an example hardware configuration of the information processing terminal 4 with reference to FIG. 6. FIG. 6 is a diagram of an example hardware configuration of the information processing terminal 4 The information processing terminal 4 includes a CPU 900, a RAM 901, and a ROM 902. These CPU 900, RAM 901, and ROM 902 are connected by a bus to which various interface circuits (input/output interface 903 and peripherals interface 904) are also connected. These interface circuits serve as an interface when the information processing terminal 4 transmits/receives data to/from various input/output devices (e.g., the electrode instrument 20A and the server 5) that are linked to the information processing terminal 4 under the control of the CPU 900.

In the electrode instrument 20A of the present embodiment, the electrically conductive cloth 23 is attached to the belt 21 in such a manner as to cover the belt 21 and the electrically insulating members 22. Alternatively, the electrode instrument 20A may, in an embodiment of the present invention, be structured such that the electrically conductive cloth 23 is attached to the belt 21 in such a manner that the electrically conductive cloth 23 can cover only a side of the belt 21 where the projections 26 are located.

In the biological information measuring system 1 of the present embodiment, the electrically conductive cloth 23 comes into contact with both front armpits of the dog. Alternatively, the biological information measuring device, in an embodiment of the present invention, may be structured such that an electrically conductive cloth comes into contact with the animal across the heart to acquire a cardiac electrical activity signal to draw an electrocardiogram. As an example, the structural member for fitting the electrode instrument 20A to the dog may be altered such that the electrically conductive cloth 23 can come into contact with the animal at two sites, one under a front armpit and the other on the groin between hind limbs. The armpits of a dog have thin hair and are suited for measurement of a cardiac electrical activity signal. As another alternative, a piece of measurement clothing as a fitting device may include projections 26 having surfaces thereof covered by an electrically conductive cloth 23 so that the electrically conductive cloth can be pressed onto the body surface of the animal due to the stretchability of the measurement clothing. This structure eliminates the need to provide the base 25, thereby reducing manufacturing cost.

The biological information measuring system 1 of the present embodiment includes the signal processing unit 3 and the information processing terminal 4 as separate units. Alternatively, the biological information measuring device, in an embodiment of the present invention, may include a terminal to be fitted to the animal, the terminal functioning as both the signal processing unit 3 and the information processing terminal 4.

### Variation Example 1

A description will be now given of electrically insulating members 22A and 22B as variation examples of the electrically insulating members 22 of Embodiment 1 with reference to FIG. 7. Portion (a) of FIG. 7 is a cross-sectional view of a structure of the electrically insulating member 22A taken parallel to a plane perpendicular to the direction in which the belt 21 is extended. Portion (b) of FIG. 7 is a cross-sectional view of a structure of the electrically insulating member 22B taken parallel to a plane perpendicular to the direction in which the belt 21 is extended.

As shown in (a) of FIG. 7, the electrically insulating member 22A includes two projections 26 projecting in different directions when viewed in a plane perpendicular to the direction in which the belt 21 is extended.

As shown in (b) of FIG. 7, the electrically insulating member 22B includes three projections 26 projecting in different directions when viewed in a plane perpendicular to the direction in which the belt 21 is extended.

The electrically insulating members 22A and the electrically insulating members 22B include a plurality of projections 26 projecting in different directions when viewed in a plane perpendicular to the direction in which the belt 21 is extended. Therefore, in the electrode instrument 20A, since the belt 21 includes a plurality of electrically insulating members 22A or 22B, the projections 26 form two or more arrays with those in different arrays projecting in different directions from the belt 21. In this structure, the electrode instrument 20A includes the projections 26 arranged in a plurality of arrays with those in different arrays projecting in different directions from the belt 21. As a result, one of the arrays of projections 26 can bring the electrically conductive cloth 23 into contact with the dog's body surface when the electrode instrument 20A is rotated around the direction in which the belt 21 is extended.

### Variation Example 2

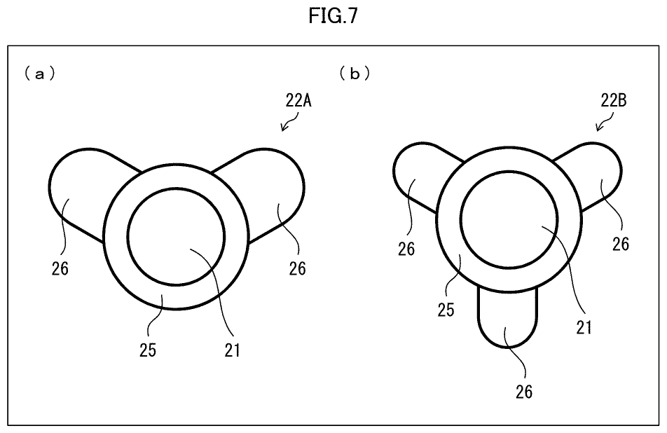

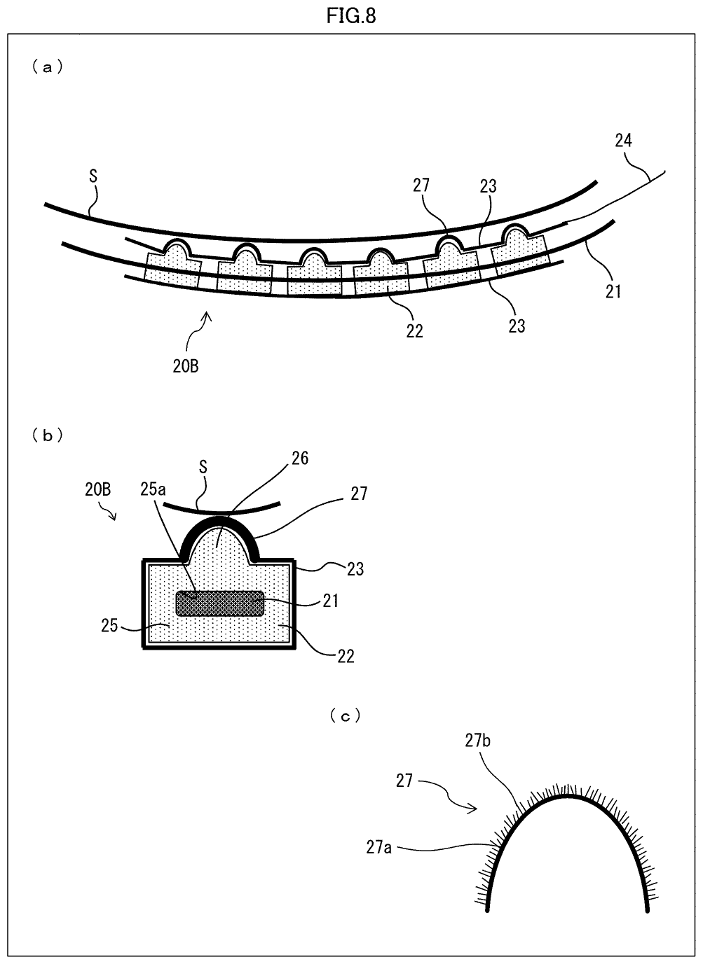

A description will be now given of an electrode instrument 20B as a variation example of the electrode instrument 20A of Embodiment 1 with reference to FIGS. 8 and 9. For convenience of description, members of Variation Example 2 that have the same function as members of the previous embodiment are indicated by the same reference numerals, and description thereof is omitted. FIG. 8 illustrates a structure of the electrode instrument 20B, (a) of FIG. 8 being a cross-sectional view of the electrode instrument 20B taken along the direction in which the belt 21 is extended, (b) of FIG. 8 being a cross-sectional view of the electrode instrument 20B taken parallel to a plane perpendicular to the direction in which the belt 21 is extended, and (c) of FIG. 8 being a cross-sectional view of a structure of a cap 27.

The electrode instrument 20B includes a belt 21, electrically insulating members 22, an electrically conductive cloth 23, a cable 24, and caps 27 as shown in (a) and (b) of FIG. 8. The belt 21 includes six electrically insulating members 22 arranged therein to form an array of projections 26 in the electrode instrument 20B as in the electrode instrument 20A.

As shown in (c) of FIG. 8, each cap 27 includes a rubber cap portion (projection) 27a and a fiber portion (electrically conductive fiber) 27b. The rubber cap portion 27a has a projection shape covering the projection 26. The rubber cap portion 27a is made of electrically conductive rubber and therefore elastic and electrically conductive. The fiber portion 27b is formed by electrostatically flocking a surface of the rubber cap portion 27a that is opposite the surface thereof covering the projection 26 with the same electrically conductive fiber as the electrically conductive cloth 23. The fiber portion 27b is therefore electrically conductive. In the electrode instrument 20B, each of the six electrically insulating members 22 has a cap 27 on the projection 26 thereof.

In the electrode instrument 20B, the electrically conductive cloth 23 is connected to an edge of the projecting cap 27. The combination of the caps 27 and the electrically conductive cloth 23 provides electrically conductive members that function as electrodes. In other words, the electrically conductive cloth 23 is provided with an opening, and the caps 27 and the electrically insulating members 22 are arranged such that the caps 27 project out of the opening. The opening has a rim thereof either in contact with or sewn to the fiber portion 27b of each cap 27, which electrically connects the cap 27 to the electrically conductive cloth 23. The caps 27, which come into contact with the dog's skin, project more from the belt 21 in this structure than in the electrode instrument 20A of Embodiment 1. As a result, the electrically conductive cloth 23 can be more easily brought into contact with the dog's skin.

The electrode instrument 20B includes the fiber portion 27b where electrically conductive fiber is implanted on the surface of the rubber cap portion 27a serving as a projection. The fiber portion 27b, electrostatically flocked with electrically conductive fiber, provides small voids on the surface thereof. The dog's hairs go into these small voids when the cap 27 is pressed onto the dog's body surface S, which facilitates the contact of the cap 27 to the dog's skin. The electrode instrument 20B is therefore more likely to successfully acquire information on the dog's cardiac electrical activity.

In addition, since the rubber cap portion 27a is elastic, the electrode instrument 20B can reduce the pressing force exerted on the dog over the pressing force exerted in a structure in which the electrically conductive cloth 23 is in contact with the electrically insulating member 22 as in the electrode instrument 20A of Embodiment 1. This mechanism can in turn further reduce damage to the dog when the electrode instrument 20B is fitted to the dog.

FIG. 9 illustrates effects of the electrode instrument 20B, (a) of FIG. 9 showing an electrocardiogram drawn based on a cardiac electrical activity signal acquired by an electrode instrument including electrically insulating members that have no projections 26 and (b) of FIG. 9 showing an electrocardiogram drawn based on a cardiac electrical activity signal acquired by the electrode instrument 20B. The graphs in FIG. 9 are prepared by amplifying a measured signal by a factor of approximately 1,000 times.

As shown in (a) and (b) of FIG. 9, the amplitude of the voltage in the electrocardiogram drawn based on a cardiac electrical activity signal acquired by the electrode instrument 20B (about 1 volt) is approximately 4 times the amplitude of the voltage in the electrocardiogram drawn based on a cardiac electrical activity signal acquired by an electrode instrument that includes electrically insulating members with no projections 26 (approximately 0.2 to 0.3 volts). Accordingly, the electrode instrument 20B is more likely to successfully acquire information on the dog's cardiac electrical activity than an electrode instrument that includes electrically insulating members with no projections 26.

### Embodiment 2

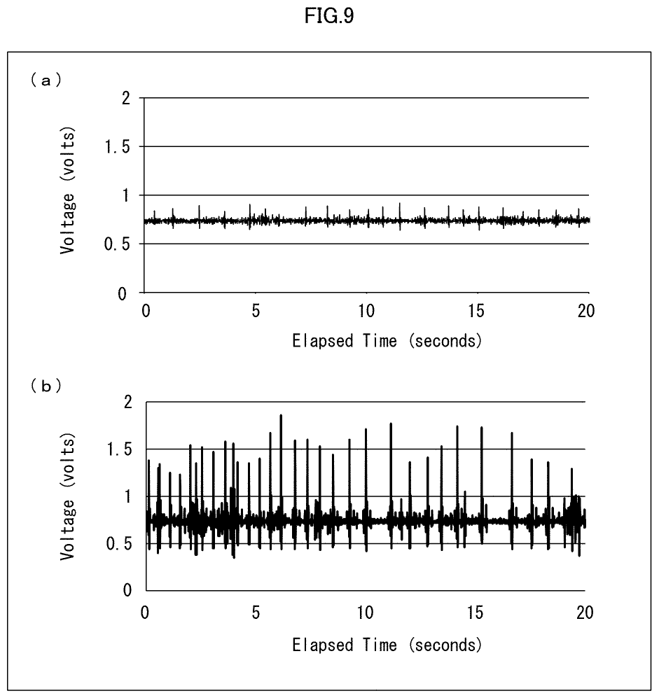

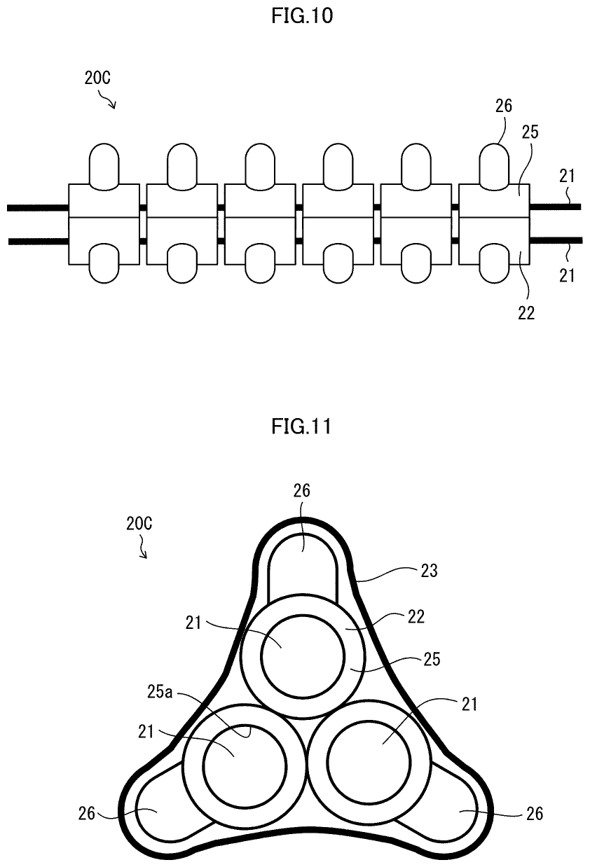

The following will describe another embodiment of the present invention with reference to FIGS. 10 and 11. The electrically conductive cloth 23 and the cable 24 are omitted from FIG. 10. The cable 24 is omitted from FIG. 11.

An electrode instrument 20C in accordance with the present embodiment differs from the electrode instrument 20A of Embodiment 1 in that the projections 26 are arranged in three arrays, each array of projections 26 projecting in a different direction from the belt 21.

FIG. 10 is a side view of the electrode instrument 20C. FIG. 11 is a cross-sectional view of the electrode instrument 20C taken parallel to a plane perpendicular to the direction in which the belt 21 is extended.

Referring to FIGS. 10 and 11, the electrode instrument 20C includes three belts (fitting devices) 21. Each belt 21 includes an array of six projections 26. In the electrode instrument 20C, each belt 21 includes electrically insulating members 22 with bases 25 in contact with the bases 25 of the electrically insulating members 22 in the other belts 21.

The electrically conductive cloth 23 in the electrode instrument 20C covers the three belts 21 and the electrically insulating members 22 included in the three belts 21. The electrically conductive cloth 23 is electrically connectable to the signal processing unit 3 via the cable 24.

As described above, the projections 26 in the electrode instrument 20C are arranged in three arrays, each array of projections 26 projecting in a different direction from the belt 21. Accordingly, the projections 26 on one of the three belts 21 can bring the electrically conductive cloth 23 into contact with the dog when the electrode instrument 20C is rotated around the direction in which the belts 21 are extended.

The projections 26 in the present embodiment are arranged in three arrays, each array of projections 26 projecting in a different direction from the belt 21. Alternatively, the electrode instrument, in an embodiment of the present invention, may include a first and a second array of projections 26, with the projections in the first array projecting in a different direction than the projections in the second array (may include projections 26 arranged in two arrays, each array of projections 26 projecting in a different direction from the belt 21). As a further alternative, the projections 26 may be arranged in four or more arrays, each array of projections 26 projecting in a different direction from the belt 21.

### Embodiment 3

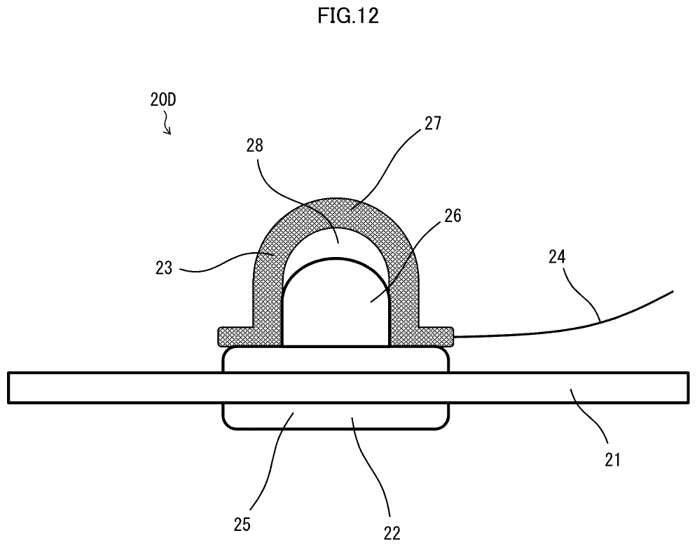

The following will describe another embodiment of the present invention with reference to FIG. 12. FIG. 12 is a cross-sectional view of an electrode instrument 20D in accordance with the present embodiment.

Referring to FIG. 12, the electrode instrument 20D includes a belt 21, an electrically insulating member 22, a cable 24, and a cap 27. In the electrode instrument 20D, the cap 27 is disposed on the electrically insulating member 22, and there is provided a gap 28 between the cap (second projection) 27 and a projection (first projection) 26 on the electrically insulating member 22. The cap 27 elastically deforms in the gap 28 when the cap 27 is pressed onto the dog's body surface. As a result, the electrode instrument 20D can reduce the pressing force exerted on the dog over the pressing force exerted in a structure in which there is provided no gap 28. This mechanism can in turn further reduce damage to the dog when the electrode instrument 20D is fitted to the dog.

### Embodiment 4

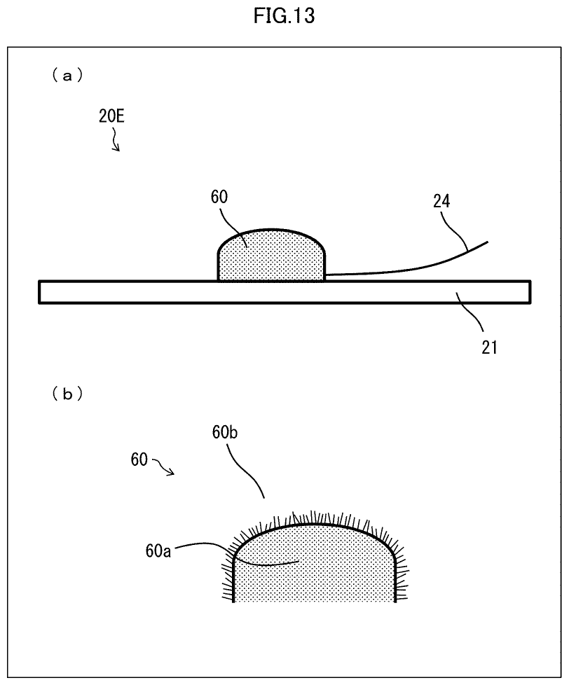

The following will describe another embodiment of the present invention with reference to FIG. 13. FIG. 13 illustrates a structure of an electrode instrument 20E in accordance with the present embodiment, (a) of FIG. 13 being a side view of the electrode instrument 20E and (b) of FIG. 13 being a cross-sectional view of a structure of a projection 60.

As shown in (a) of FIG. 13, the electrode instrument 20E includes a belt 21, a projection 60, and a cable 24.

As shown in (b) of FIG. 13, the projection 60 includes a rubber portion 60a and a fiber portion (electrically conductive fiber) 60b. The projection 60 functions as an electrode for the electrode instrument 20E.

The rubber portion 60a is made of electrically conductive rubber and attached to the belt 21 using an adhesive so as to project from the belt 21. Since the rubber portion 60a is elastic, the rubber portion 60a can reduce the pressing force exerted on the dog. This mechanism can in turn further reduce damage to the dog when the electrode instrument 20E is fitted to the dog.

The fiber portion 60b is formed by electrostatically flocking a surface of the rubber portion 60a with the same electrically conductive fiber as the electrically conductive cloth 23. The fiber portion 60b is therefore electrically conductive. The fiber portion 60b, formed by electrostatic flocking with electrically conductive fiber, provides small voids on the surface thereof. The dog's hairs go into these small voids when the projection 60 is pressed onto the dog's body surface, which facilitates the contact of the projection 60 with the dog's skin. The electrode instrument 20E is therefore more likely to successfully acquire information on the dog's cardiac electrical activity.

The cable 24 is for transmitting the dog's cardiac electrical activity signal acquired by the projection 60 to the signal processing unit 3. The cable 24 has an end thereof connected to the projection 60 and the other end thereof connected to the signal processing unit 3, thereby electrically connecting the projection 60 to the signal processing unit 3.

Since the electrode instrument 20E includes the projection 60 functioning as an electrode, there is no need to provide the electrically insulating member 22 as in the electrode instruments of the other embodiments, which can reduce manufacturing cost.

The belt 21 may be made of an electrically conductive material so that the projection 60 and the signal processing unit 3 can be electrically connected via the belt 21. This structure eliminates the need to provide the cable 24, thereby reducing manufacturing cost.

The present invention is not limited to the description of the embodiments above and may be altered within the scope of the claims. Embodiments based on a proper combination of technical means disclosed in different embodiments are encompassed in the technical scope of the present invention. Furthermore, a new technological feature can be created by combining different technological means disclosed in the embodiments.

### CROSS-REFERENCE TO RELATED APPLICATIONS

The present application claims the benefit of priority to Japanese Patent Application, Tokugan, No. 2017-035076, filed Feb. 27, 2017, the entire contents of which are incorporated herein by reference.

### REFERENCE SIGNS LIST

1 Biological Information Measuring System

2 Measuring Unit

3 Signal Processing Unit

3a Housing

4 Information Processing Terminal

5 Server

11 Neck Belt

12 Trunk Belt

20A to 20E Electrode Instrument

21 Belt (Fitting Device)

22, 22A, 22B Electrically Insulating Member

23 Electrically Conductive Cloth (Electrically Conductive Fiber)

24 Cable

25 Base

25a Through Hole

26, 60 Projection (First Projection)

27 Cap (Second Projection)

27a Rubber Cap Portion (Projection)

27b, 60b Fiber Portion (Electrically Conductive Fiber)

28 Gap

31 Signal Preprocessing Unit

32 Transmitting Unit

41 Receiving Unit

42 Control Unit

43 Memory Unit

44 Display Unit

45 Communications Unit

60a Rubber Portion (Projection)

S Body Surface

1: An electrode instrument that acquires biological information, the instrument comprising:
a fitting device configured to fit the electrode instrument to a living organism;
a projection projecting from the fitting device; and
electrically conductive fiber disposed on a surface of the projection or in a vicinity thereof to function as a part of an electrode.

2: The electrode instrument according to claim 1, wherein the electrically conductive fiber is implanted in the surface of the projection.

3: The electrode instrument according to claim 1, wherein:
the projection includes a first projection and a second projection positioned to cover the first projection;
the second projection is elastic and has the electrically conductive fiber disposed on a surface thereof; and
the first projection and the second projection form a gap therebetween.

4: The electrode instrument according to claim 1, wherein the electrically conductive fiber is shaped as a piece of cloth covering the projection.

5: The electrode instrument according to claim 1, the projection comprising a plurality of projections projecting in different directions.

6: The electrode instrument according to claim 1, the projection comprising an array of projections.

7: The electrode instrument according to claim 6, the array of projections comprising a first array of projections and a second array of projections, wherein the projections in the first array project in a different direction than the projections in the second array.

8: The electrode instrument according to claim 1, wherein the projection(s) has/have such hardness that the projection(s) does/do not deform when pressed onto the living organism.

9: A biological information measuring device comprising the electrode instrument according to claim 1.
